(19) United States
(12) Patent Application Publication
(10) Pub. No.: US 2025/0262176 A1
(43) Pub. Date: Aug. 21, 2025

(54) Conditioning Irradiated Tissue for Increasing Vascularity

(71) Applicant: The Board of Trustees of the Leland Stanford Junior University, Stanford, CA (US)

(72) Inventors: Derrick C. Wan, Stanford, CA (US); Geoffrey C. Gurtner, Portola Valley, CA (US); Michael T. Longaker, Atherton, CA (US)

(21) Appl. No.: 19/186,323

(22) Filed: Apr. 22, 2025

Related U.S. Application Data
(62) Division of application No. 17/721,125, filed on Apr. 14, 2022, now Pat. No. 12,303,475.
Application No. 17/721,125 is a division of application No. 16/642,467, filed on Feb. 27, 2020, now Pat. No. 11,331,288.
(60) Provisional application No. 62/558,698, filed on Sep. 14, 2017.
(60) Provisional application No. 62/588,230, filed on Nov. 17, 2017.

(51) Int. Cl. A61K 31/16; A61K 9/00; A61K 9/107

(52) U.S. Cl. CPC A61K 31/16; A61K 9/0019; A61K 9/1075

(57) ABSTRACT

Methods of increasing perfusion and vascularity in irradiated tissue and of increasing retention of fat cells in a fat graft in irradiated tissue by applying an effective amount of DFO to the irradiated tissue at a treatment site. The DFO may be administered transdermally.

### CROSS REFERENCE TO RELATED APPLICATIONS

This application is a U.S. National Stage Application of PCT Application No. PCT/US2018/050626 filed Sep. 12, 2018, which application claims the benefit of U.S. Application No. 62/558,698, filed Sep. 14, 2017, and U.S. Application No. 62/588,230, filed Nov. 17, 2017, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### STATEMENT AS TO FEDERALLY SPONSORED RESEARCH

This invention was made with Government support under Grant Nos. DE021683, DE025597, DE026914 and DE024269 awarded by the National Institutes of Health. The Government has certain rights in the invention.

### BACKGROUND

After heart disease, cancer remains the leading cause of death in the United States, with an estimated 1.6 million new cancer cases diagnosed and 600,000 cancer-related deaths projected for 2017. However, in recent years, substantial progress in medical care has been made, with surgery, chemotherapy, and radiation therapy increasing both the number of cancer survivors and the length of their survival. With this improvement, long-term issues related to treatment of cancer, such as with radiation therapy, have become increasingly apparent, and have been shown to profoundly impact quality of life. Radiation-induced soft tissue injury is one of the most common side effects of radiotherapy, affecting over 90% of patients, and the resulting soft tissue atrophy and fibrosis can lead to both severe cosmetic and long-term functional impairment.

Radiation therapy is a mainstay in the treatment of many malignancies. Radiation therapy can cause collateral damage to surrounding tissue, however, with resultant hypovascularity, fibrosis, and atrophy, and the damaged tissue can be difficult to reconstruct. Fat grafting has been shown to improve the quality of irradiated skin, but volume retention of the grafted fat cells is significantly decreased.

Chronic radiation injury is characterized by epidermal thinning, eosinophilic homogenized sclerosis of dermal collagen, scattered large and atypical fibroblasts, and fibrous thickening with luminal obliteration of deep vessels. Vascular damage and development of fibrosis is thought to result from radiation-induced cytokine expression, generation of reactive oxygen species, and cellular apoptosis, and soft tissue reconstruction of such hostile sites remains extremely challenging. While autologous fat grafting has become increasingly popular to address post-radiation soft tissue deficits, fibroinflammatory changes and hypovascularity have been associated with poorer fat graft outcomes. Improved retention has been noted with cell assisted lipotransfer, but the functional heterogeneity among stromal cells used to enrich lipoaspirate, in concert with concerns regarding post-oncologic locoregional recurrence, has limited the wide-spread adoption of this strategy.

Over 5.6 million soft tissue reconstructive procedures are performed annually in the United States, with the majority related to tumor extirpation and sequelae of adjuvant radiation therapy. Even with intact overlying epithelium, insufficient underlying soft tissue results in visible asymmetry and contour abnormalities, and may also contribute to unstable wounds and inadequate protection of critical organs and structures including bone, implanted hardware, and large vessels. While radiation therapy has been shown to be incredibly effective at reducing local recurrence risk for various tumors, collateral damage to adjacent soft tissue resulting in obliteration of microvasculature and fibrosis may significantly complicate reconstructive strategies.

Autologous fat grafting has emerged as an increasingly popular strategy to manage soft tissue deficiencies throughout the body, and this technique has been applied clinically to reconstruct defects secondary to cancer resection. However, transfer of avascular fat to irradiated sites remains challenging, as radiation induced changes to the recipient bed (i.e., hypovascularity) result in poorer fat graft survival. Cell-based strategies to enhance volume retention, through enrichment of fat grafts with additional adipose-derived stromal cells, have been shown to improve outcomes, but translation of this approach remains difficult due to known heterogeneity of the stromal vascular fraction and regulatory hurdles.

Deferoxamine (DFO) is an FDA-approved iron chelating medication for acute iron intoxication and chronic iron overload that has also been shown to increase angiogenesis. DFO has been demonstrated to increase hypoxia-inducible factor-1 alpha (HIF-1α) activity and enhance expression of angiogenic growth factors. Studies have also shown local injection of DFO to improve ischemic flap survival in both mouse and pig models, with increased skin flap blood perfusion and capillary density noted in DFO-treated animals. Furthermore, in the setting of irradiated bone, multiple reports have found DFO to promote bone regeneration following distraction osteogenesis through enhanced vascularity.

The potential of DFO as an angiogenic and antioxidant agent with the potential to improve fat graft survival in healthy subjects has also been studied, and its use to increase the viability of fat grafts for plastic surgery has been proposed. Importantly, DFO was recently suggested to promote fat graft viability in a rat model. Temiz and colleagues described serial injections of 300 mg DFO into inguinal fat pads transplanted to parascapular subcutaneous pockets, which resulted in significantly greater weight measurements after two months. However, more inflammation and fibrosis was noted in DFO injected fat grafts, though no change in cellular apoptosis was appreciated. Repeated manipulation of fat grafts with each injection may have contributed to this observation. In addition, adipogenic differentiation of resident stromal cells has been purported to contribute to long-term fat graft retention, and direct exposure of DFO to fat grafts may be detrimental to this process. Studies have shown intracellular iron deficiency through DFO administration to severely blunt adipocyte differentiation. These findings thus temper enthusiasm for direct injection of DFO into fat grafts.

### SUMMARY OF THE DISCLOSURE

One aspect of the invention provides a method of increasing retention of fat cells in a fat graft in irradiated tissue. In some embodiments, the method includes the steps of applying an effective amount of DFO to the irradiated tissue at a treatment site; and increasing retention of fat cells in the fat graft.

In some embodiments, the applying step includes the step of injecting DFO under dermis at the treatment site. In some embodiments, the applying step includes the step of applying DFO transdermally at the treatment site, such as by applying a transdermal delivery device to a tissue surface at the treatment site, with the DFO optionally being encapsulated in reverse micelles. In any of these embodiments, the applying step may include the step of applying DFO to the irradiated tissue in multiple discrete doses.

Some or all of the preceding embodiments may include the step of grafting fat cells at the treatment site after the applying step.

Another aspect of the invention provides a method of increasing blood perfusion in irradiated tissue. In some embodiments, the method includes the steps of applying an effective amount of DFO to the irradiated tissue at a treatment site; and increasing blood perfusion of the irradiated tissue at the treatment site.

In some embodiments, the applying step includes the step of injecting DFO under dermis at the treatment site. In some embodiments, the applying step includes the step of applying DFO transdermally at the treatment site, such as by applying a transdermal delivery device to a tissue surface at the treatment site, with the DFO optionally being encapsulated in reverse micelles. In any of these embodiments, the applying step may include the step of applying DFO to the irradiated tissue in multiple discrete doses.

Yet another aspect of the invention provides a method of increasing vascularity in irradiated tissue. In some embodiments, the method includes the steps of applying an effective amount of DFO to the irradiated tissue at a treatment site; and increasing vascularity of the irradiated tissue at the treatment site.

In some embodiments, the applying step includes the step of injecting DFO under dermis at the treatment site. In some embodiments, the applying step includes the step of applying DFO transdermally at the treatment site, such as by applying a transdermal delivery device to a tissue surface at the treatment site, with the DFO optionally being encapsulated in reverse micelles. In any of these embodiments, the applying step may include the step of applying DFO to the irradiated tissue in multiple discrete doses.

Another aspect of the invention provides a method of increasing collagen deposition in skin in irradiated tissue. In some embodiments, the method includes the steps of applying an effective amount of DFO to the irradiated tissue at a treatment site; and increasing collagen deposition of the irradiated tissue at the treatment site.

In some embodiments, the applying step includes the step of injecting DFO under dermis at the treatment site. In some embodiments, the applying step includes the step of applying DFO transdermally at the treatment site, such as by applying a transdermal delivery device to a tissue surface at the treatment site, with the DFO optionally being encapsulated in reverse micelles. In any of these embodiments, the applying step may include the step of applying DFO to the irradiated tissue in multiple discrete doses.

Still another aspect of the invention provides a method of reducing stiffness in irradiated tissue, as measured by stress/strain curves (Youngs Modulus). In some embodiments, the method includes the steps of applying an effective amount of DFO to the irradiated tissue at a treatment site; and reducing stiffness of the irradiated tissue at the treatment site.

In some embodiments, the applying step includes the step of injecting DFO under dermis at the treatment site. In some embodiments, the applying step includes the step of applying DFO transdermally at the treatment site, such as by applying a transdermal delivery device to a tissue surface at the treatment site, with the DFO optionally being encapsulated in reverse micelles. In any of these embodiments, the applying step may include the step of applying DFO to the irradiated tissue in multiple discrete doses.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a schematic of an irradiated tissue treatment according to this invention.

FIG. 2A shows representative photos of heat maps of mice scalps before irradiation, after irradiation, and after treatment with either saline or DFO. Darker areas represent lower perfusion and lighter areas represent higher perfusion.

FIG. 2B shows a quantification of laser Doppler perfusion index from the irradiated mouse scalps. DFO treatments (T) caused a significance rise in perfusion after 4 treatments (T4), compared to saline injection, and plateaued after 5 treatments (T5) (*p<0.05).

FIGS. 3A-B are representative three-dimensional reconstructions of fat grafts after eight weeks in either DFO (FIG. 3A) or saline (FIG. 3B) preconditioned irradiated scalp.

FIG. 3C shows that quantification of fat graft volumes revealed significantly increased retention in fat grafts placed into DFO treated scalp (upper line) when compared to saline treated scalp (lower line) after six and eight weeks (*p<0.05).

FIG. 4A-E show a histologic evaluation of irradiated scalp vascularity with representative images taken at 20× magnification of scalp skin with immunofluorescent staining for CD31 showing increased vascularity with DFO preconditioning. Scale bar represents 100 μm.

FIG. 5 shows a quantification of CD31 immunofluorescent staining revealed significant drop following irradiation. Significant improvement was noted with DFO treatment, and vascularity was further enhanced with fat grafting (*p<0.05).

FIG. 6 shows a Laser Doppler Analysis following fat grafting. FIG. 6A shows representative LDA images of saline (top) and DFO (bottom) treated tissue scalp following fat grafting. Darker areas represent lower perfusion and lighter areas represent higher perfusion. In FIG. 6B, a quantification of laser Doppler perfusion index demonstrated DFO treated scalp (upper line) had significantly higher perfusion than saline treated scalp (lower line) two weeks after fat grafting (*p<0.05). Both groups demonstrated increased perfusion after fat grafting with no significant difference appreciated after week 2.

FIG. 7-10 show an evaluation of irradiated scalp histology following fat grafting. FIG. 7A-E show representative H&E stained sections at 10× magnification of non-irradiated, healthy skin, irradiated skin after saline or DFO treatment, and irradiated skin after saline or DFO treatment and fat grafting. Scale bar represents 200 μm.

FIG. 8 shows that a quantification of dermal thickness demonstrated significant increase following radiation, with no difference between saline or DFO treated skin. Both treatment groups demonstrated significant reduction following fat grafting (*p<0.05).

FIGS. 9A-E show representative picrosirius red stained sections at 20× magnification demonstrating density of positive-stained collagen after irradiation and saline or DFO preconditioning, followed by fat grafting. Scale bar represents 100 μm.

FIG. 10 shows that a quantification of collagen content revealed significant increase in collagen following radiation, irrespective of saline or DFO treatment. Both groups demonstrated significant reduction following fat grafting (*p<0.05).

FIG. 11 shows that quantification of laser Doppler perfusion index demonstrated scalp pretreated with a DFO transdermal delivery system (upper line) had significantly higher perfusion than scalp pretreated with a transdermal delivery system lacking DFO (lower line) one week after fat grafting (*p<0.05).

FIG. 12 shows representative LDA images of mice scalp showing perfusion in mice prior to radiation therapy (leftmost image), in an experimental group pretreated with DFO delivered by a transdermal delivery device (upper two images), and in a control group pretreated with transdermal delivery devices without DFO (lower two images) immediately after cessation of radiation therapy and one week later.

FIG. 13 shows that quantification of fat graft volumes revealed significantly increased retention in fat grafts placed into DFO treated scalp (upper line) when compared to control (no DFO) treated scalp (lower line) after one and two weeks.

FIG. 14 shows representative three-dimensional reconstructions of fat grafts after two weeks in either DFO (upper) or control (lower) preconditioned irradiated scalp.

FIGS. 15A-C and 16 show skin stiffness data for healthy mice, mice treated with transdermal DFO delivery devices and mice pretreated with transdermal delivery devices without DFO.

### DETAILED DESCRIPTION

The present invention provides a method to improve fat graft outcomes. One aspect of the invention provides a method of preconditioning irradiated soft tissue site with DFO to enhance vascularity prior to implantation of a fat graft. HIF-la is typically degraded by prolyl hydroxylase domain-containing protein 2 (PHD2). DFO, through chelation of the iron co-factor for PHD2 activity, has been shown to stabilize HIF-1α, leading to an increase in downstream angiogenic factors and recruitment of endothelial progenitor cells. This is the mechanism by which DFO has been thought to promote revascularization of ischemic skin flaps, enhance wound healing in diabetic mice, and augment callus size, mineralization, and mechanical strength at irradiated bone injury sites. Furthermore, reversal of radiation induced hypovascularity has also been appreciated with DFO treatment during mandibular distraction osteogenesis. All of these findings support the potential for DFO, through stabilization of HIF-1α and increased angiogenic gene expression, to precondition the irradiated recipient site for subsequent fat grafting.

Preconditioning the irradiated tissue at the fat graft site with DFO before implantation of the fat graft facilitates earlier revascularization of the fat graft. Histologic analysis of treated skin according to this method has revealed increased vascularity, which translates to enhanced volume retention, when fat grafts were placed into DFO preconditioned recipient sites. Interestingly, the addition of fat grafts to DFO treated irradiated tissue leads to further improvement in vascularity, even though DFO-related effects might plateau after four treatments. This suggests that alternative mechanisms may also be employed by transferred adipocytes and associated stromal cells to improve vascularity following fat grafting. Finally, the effects of DFO treatment on skin vascularity are not associated with significant changes to dermal thickness and collagen content, compared to decreased dermal thickness and collagen content following fat grafting. The architectural changes observed in the dermis with decreased collagen secondary to fat transfer may not necessarily be a result of improved vascularity alone.

In patients with radiation fibrosis and soft tissue atrophy, preconditioning tissue with serial DFO injections prior to fat grafting may be difficult logistically and not well tolerated by patients. Transdermal delivery of DFO to irradiated tissue prior to and/or after fat graft implantation may be used as an alternative to delivery of DFO via direct injection. Such an approach may also be potentially effective at preconditioning irradiated tissue for fat grafting and would likely be better tolerated by patients. Alternatively, nanoparticle formulations of DFO have also been developed, and their controlled release of DFO may similarly be employed to improve vascularity of irradiated skin. These nanoparticles may also be directly injected with fat grafts to promote earlier revascularization.

As DFO promotes expression of multiple angiogenic factors through stabilization of HIF-1α, concern may also be raised regarding its use in irradiated post-oncologic resection sites. Though no studies, to our knowledge, have demonstrated an increased risk for cancer recurrence following local administration of DFO, several reports have suggested an anti-tumor effect. Iron is necessary for oxygen transport, cell metabolism, and growth, and it is especially important in cells with active growth. Not surprisingly, iron chelators such as DFO have been found to reduce liver fibrosis, and its effect on iron metabolism has been shown to clinically reduce progression of hepatocellular carcinoma. Iron dependency has also been reported in human epidermal growth factor receptor 2 positive breast cancer cells, and multiple breast cancer cell lines have been shown to be vulnerable to iron chelation. These reports thus suggest local DFO application may not be associated with increased risk for cancer recurrence.

DFO treatment can improve radiation-induced hypovascularity, and this enhanced perfusion may improve the quality of the recipient site for fat grafting. Following DFO treatment, long-term retention of fat grafts injected into irradiated sites was significantly improved.

Reconstruction of irradiated tissue remains challenging owing to radiation induced alterations to the recipient bed. Fibroinflammatory changes and hypovascularity have been shown to impact fat graft retention, and while cell-based strategies have been shown to improve outcomes, regulatory and safety concerns have, to date, limited their translational potential. As an alternative approach, preconditioning irradiated tissue with deferoxamine improves local perfusion, which is associated with improved radiographic and histologic fat graft outcomes. Preconditioning with deferoxamine prior to fat grafting therefore holds promise for enhancing reconstruction outcomes for irradiated tissue.

### EXAMPLE 1

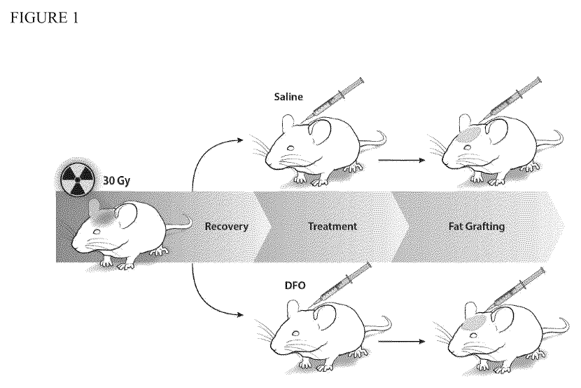

Adult 60-day-old male Crl:NU-Fox1^{NU} immunocompromised mice were used for experiments in this study. Twelve mice were treated with a total of 30 Gy external beam radiation, delivered as six fractionated doses of 5 Gy each over 12 days, followed by 5 weeks of recovery. An additional six non-irradiated mice were used as healthy controls for laser Doppler analysis (LDA) and skin analysis. Irradiated mice were divided into two treatment groups: a DFO experimental group and saline control group. Following recovery, mice underwent injection of either DFO (1 mg in 100 μl saline) or 100 μl of saline alone beneath the dermis every other day for a total of seven treatments. FIG. 1 shows a schematic of this irradiated scalp treatment.

After irradiation, fat grafting was performed on the irradiated mice. After informed consent was obtained, lipoaspirate was obtained from three healthy female donors, ages 45, 49 and 51, with no other medical co-morbidities under an approved IRB protocol #2188. Lipoaspirate was allowed to settle for 15 minutes for layers to separate by gravity sedimentation, and then oil and blood layers were removed by vacuum aspiration. The remaining fat layer was centrifuged at 1300 rcf for 3 minutes at 4° C. Any remaining oil and blood was again removed and the remaining fat was transferred into 1cc syringes for injection through a 14-gauge needle. Fat grafting was performed beneath the scalp by creating a subcutaneous tunnel with the needle and then injecting 200 μl of lipoaspirate in retrograde fashion while pulling the needle out.

Laser Doppler Analysis (“LDA”) was performed to measure perfusion at the irradiated site using a Perimed PIM 3 laser Doppler perfusion imager (Datavagen, Sweden). The signal generated by the LDA, laser Doppler perfusion index (LDPI), was used for comparative purposes. LDPI is a product of the blood cell velocity and concentration and is represented by a color spectrum, with black/dark blue representing low perfusion and red representing high perfusion. LDA was performed prior to irradiation, following completion of irradiation and recovery, and then twenty-four hours following each treatment with DFO or saline. LDA was also performed every two weeks after fat grafting.

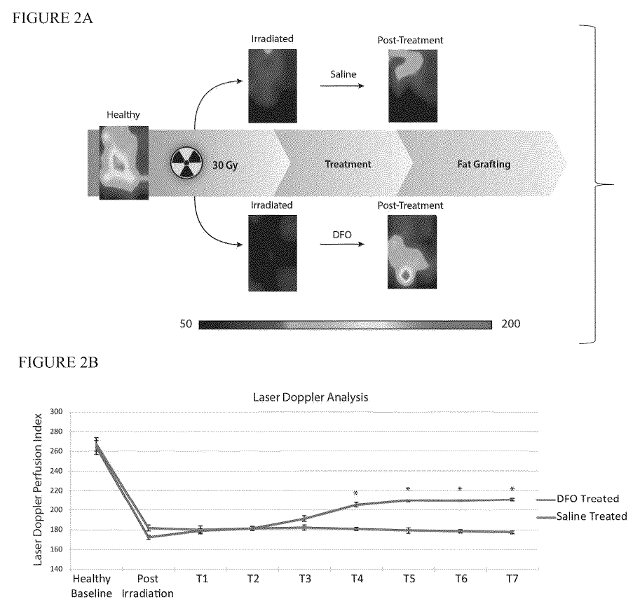

Five images were taken of each mouse, and the average LDPI of the five images was recorded. FIG. 2A shows representative photos of heat maps of mice scalps before irradiation, after irradiation, and after treatment with either saline or DFO. Darker areas represent lower perfusion and lighter represent higher perfusion. FIG. 2B shows that quantification of laser Doppler perfusion index demonstrated a significant decrease in perfusion after irradiation. Laser Doppler analysis shows improved perfusion of irradiated tissue with DFO treatment. Laser Doppler analysis allows for the estimation of in vivo local blood perfusion in the microcirculation through frequency shifts in light that has been scattered by moving red blood cells. This facilitated longitudinal measurements in the same animal following each treatment with DFO. DFO treatments (T) (upper line in FIG. 2B) caused a significance rise in perfusion after 4 treatments (T4), compared to saline injection (lower line in FIG. 2B), and plateaued after 5 treatments (T5) (*p<0.05).

Mice were also imaged using a MicroCAT-II in vivo X-Ray micro-CT scanner (Imtek, Inc.; Knoxville, TN) two days after fat graft injection for baseline volume measurements. Fat graft volume retention was then analyzed every two weeks over a total of 8 weeks using microcomputed tomography, and images were reconstructed as three-dimensional surfaces through cubic-spline interpolation. All reconstructions were performed by a single investigator to avoid inter-observer variability.

For skin analysis, scalp skin biopsy was harvested from both treatment groups following completion of radiation and then 8 weeks following fat grafting. Specimens were fixed in 4% paraformaldehyde, processed, and embedded in paraffin for sectioning. For dermal thickness measurement, sections were stained with hematoxylin and eosin (H&E) and imaged using a Leica DM5000 B Light microscope (Leica Microsystems; Buffalo Grove, IL) at the 20× objective. Dermal measurements were made on ten stained sections from each sample. Picrosirius red staining was also performed for collagen content. Vascularity was determined with CD31 immunoflourescent staining (1:100 Ab28364; Abcam; Cambridge, MA and 1:200 AF547; Thermo Fisher Scientific; Waltham, MA) and DAPI counterstaining to visualize cell nuclei. Fluorescent images were obtained using an X-Cite 120 Fluorescence Illumination System (Lumen Dynamics Group, Inc.; Ontario, Canada) at the 20× objective. Quantification of CD31 staining was performed using ImageJ (National Institutes of Health; Bethesda, MD), with pixel-positive area per high power field measured to determine vascular density (11). Comparisons for both dermal thickness and CD31 immunofluorescent staining were also made to non-irradiated skin.

Following completion of irradiation and five-week recovery, perfusion at the scalp was noted to significantly drop from 265.23±7.01 LDPI (pre-radiation baseline) to 176.70±2.59 LDPI (FIG. 2B). However, treatment of the scalp with 1 mg of DFO every other day after radiation recovery resulted in increased LDPI, which became significant after four treatments (205.08±2.30 LDPI) (*p<0.05). No increase in LDPI measurements was noted after four treatments, though, as three additional treatments of DFO did not result in any significant increase to perfusion. In contrast, injection of saline alone resulted in no change to LDPI measurements over the entire treatment course, as shown by the lower line in FIG. 2B.

For the statistical analysis, data are presented as means±SE. Two-tailed Student's t-test was used for comparison between two groups and an analysis of variance with Tukey post-hoc test was used for multiple group comparisons. All analyses were performed using StatPlus software (Analyst-Soft, Inc., Alexandria, Va.). A value of *p<0.05 was considered significant.

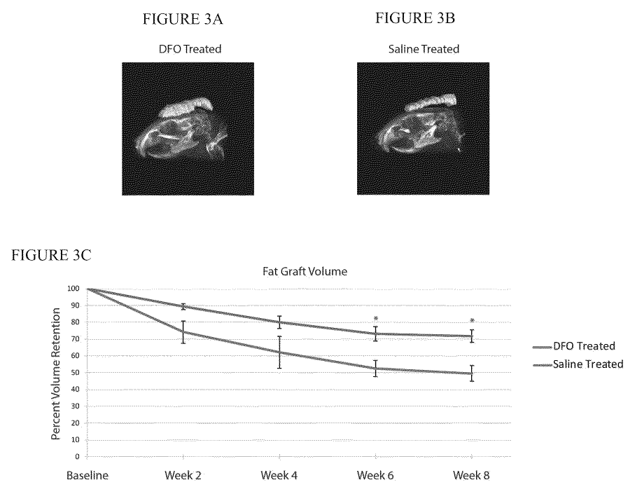

In vivo radiographic analysis of fat grafts showed DFO preconditioned irradiated mice retained more fat volume (89.24%+1.69) after two weeks compared to saline injected control mice (74.03+7.91) (FIGS. 3A-C). Fat graft volume retention was consistently greater among DFO treated mice (upper line in FIG. 3C) compared to saline control mice (lower line in FIG. 3C), and at 6 and 8 weeks, these differences were statistically significant (week 6: 73.17%±4.26 DFO vs. 52.40%±4.83 saline treated, and week 8: 71.75%±3.70 DFO vs. 49.47%+4.62 saline treated; *p<0.05).

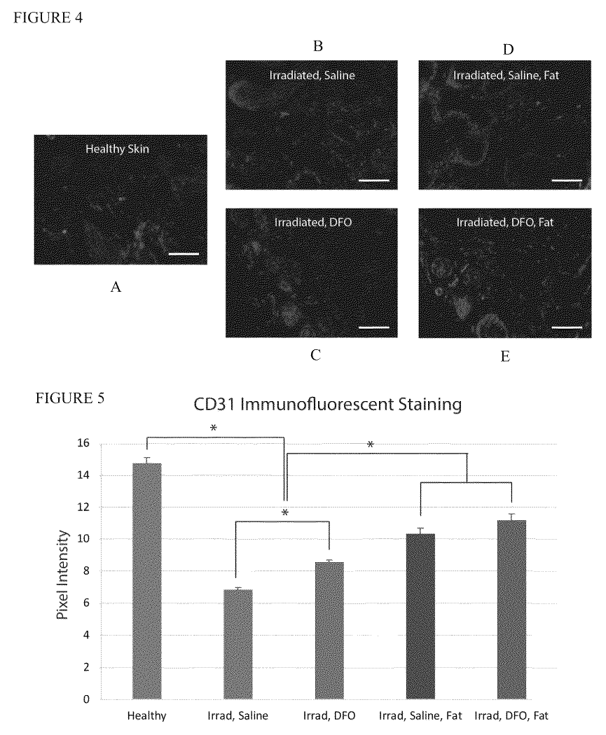

Following irradiation and saline control treatment, vascularity in skin biopsies, as demonstrated by CD31 staining, was found to be significantly lower than non-radiated healthy skin (*p<0.05) (FIG. 4A-E and FIG. 5). However, treatment of irradiated skin with DFO resulted in increased CD31 staining, though this did not reach healthy skin levels, as shown in FIG. 4A. As expected following fat grafting, skin biopsies obtained after 8 weeks also demonstrated increased CD31 staining compared to control, saline injected irradiated skin. Interestingly, slightly more CD31 staining following fat grafting was also noted with DFO preconditioned mice relative to saline control fat grafted mice, though this difference was not significant.

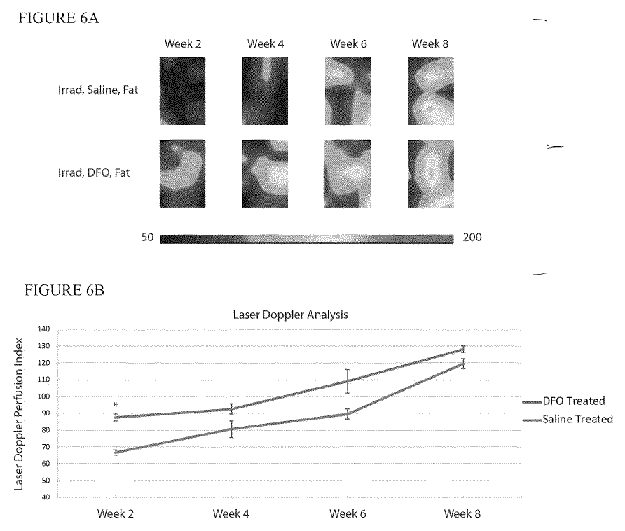

Perfusion of the skin following fat grafting was also measured by LDA, and LDPI values were found to be lower than immediately following completion of DFO or saline preconditioning due to changes in three-dimensional architecture of the region of interest following placement of fat. However, two weeks following injection of fat grafts, significantly more perfusion was still noted in DFO preconditioned mice (86.33±2.00 vs. 65.72±2.02 LDPI for saline control; *p<0.05) (FIG. 5). Perfusion also continued to increase in the DFO preconditioned mice following fat grafting (upper line in FIG. 6B), but perfusion similarly increased in saline injected control mice after fat grafting (lower line in FIG. 6B), and after week 2, no significant difference on LDA was appreciated between the two groups (127.78±2.29 vs. 119.18±4.09 LDPI for DFO and saline treated mice eight weeks following grafting, respectively; p>0.05) (FIGS. 6A-B).

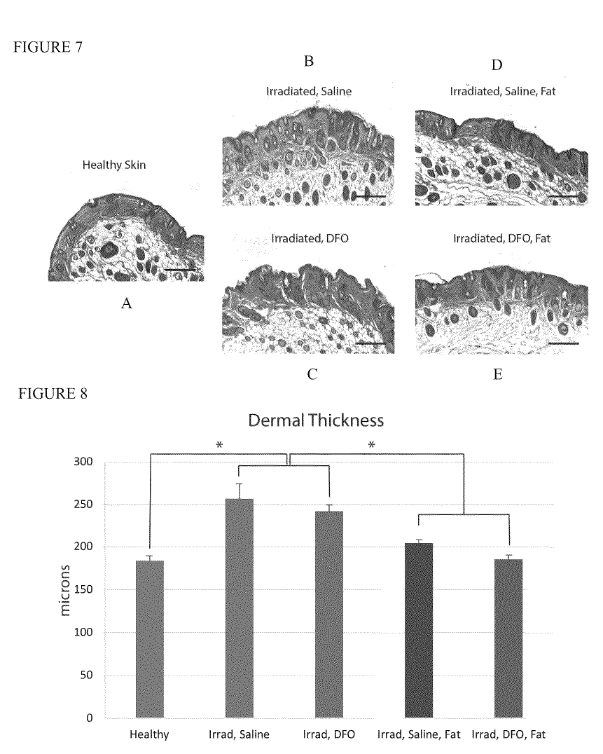

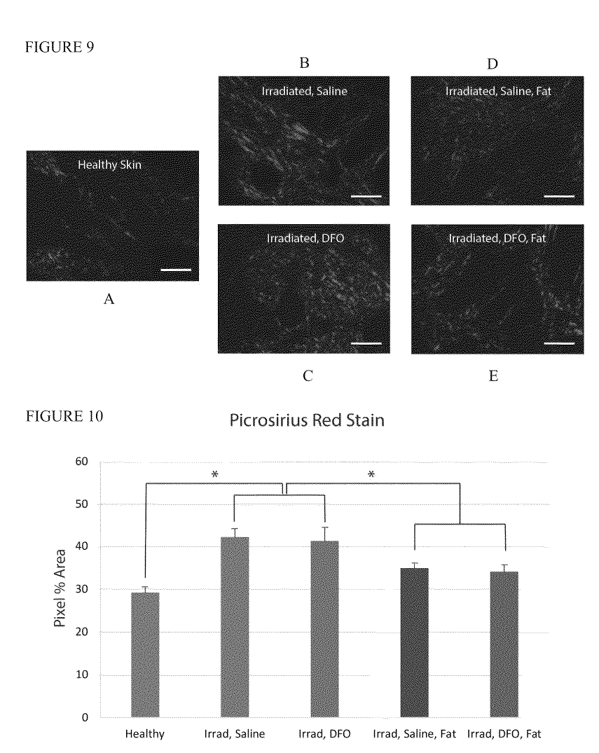

Finally, dermal thickness of irradiated skin following saline treatment was significantly greater than healthy, non-irradiated skin (*p<0.05) (FIGS. 7-8). Compared to saline injected mice (256.71±16.76 pm), DFO treatment on irradiated skin resulted in a slight decrease in dermal thickness, (242.09±7.22 pm) but this was not significantly less. However, fat grafting, whether into saline or DFO preconditioned sites, was found to significantly decrease dermal thickness, though there was no significant difference when comparing these two groups (p>0.05). Paralleling these findings, picrosirius red staining revealed significantly increased collagen content following irradiation and saline treatment (*p<0.05). DFO treatment on irradiated skin resulted in a slight decrease in collagen content which was not statistically significant. And similar to our observations with dermal thickness, fat grafting, whether into saline or DFO preconditioned sites, was found to significantly reduce collagen content (*p<0.05) (FIGS. 9-10).

Thus, local injections of DFO into irradiated hypovascular skin improved perfusion, as measured by laser Doppler analysis. Laser Doppler analysis allowed for the estimation of in vivo local blood perfusion in the microcirculation through frequency shifts in light that has been scattered by moving red blood cells. This facilitated longitudinal measurements in the same animal following each treatment with DFO. Histologic analysis of treated skin also revealed increased vascularity by CD31 staining following DFO treatment. This translated to enhanced volume retention when fat grafts were placed into DFO preconditioned recipient sites. Interestingly, the addition of fat grafts to DFO treated irradiated tissue led to further improvement in vascularity, even though DFO-related effects were seen to plateau after four treatments. This suggests that alternative mechanisms may also be employed by transferred adipocytes and associated stromal cells to improve vascularity following fat grafting. Finally, the effects of DFO treatment on skin vascularity were not found to be associated with significant changes to dermal thickness and collagen content.

### EXAMPLE 2

Adult 60-day-old male Crl:NU-Fox1NU immunocompromised mice were used for experiments in this study. Twelve mice were treated with a total of 30 Gy external beam radiation, delivered as six fractionated doses of 5 Gy each every other day over 12 days, followed by one month of recovery. An additional six non-irradiated mice were used as healthy controls for laser Doppler analysis (LDA) and skin analysis. Irradiated mice were divided into two treatment groups: a DFO experimental group and a control group. Following recovery, the we applied to the irradiated scalp skin of the DFO experimental group a transdermal delivery system comprising a dry film having DFO at a concentration of 13.4% weight/weight % of film encapsulated in a reverse micelle with a non-ionic surfactant stabilized by polyvinylpyrrolidine (PVP) in an ethylcellulose matrix, cut into a ⅝ inch circle and covered by a silicon sheet of the same size. Identical transdermal delivery devices, but omitting the DFO, were applied to the irradiated scalp skin of the control group mice. The transdermal delivery systems were left in place for two days, then replaced with new devices. After irradiation and treatment with seven changes of the transdermal delivery devices, fat grafting was performed on the irradiated mice, as described in Example 1 above.

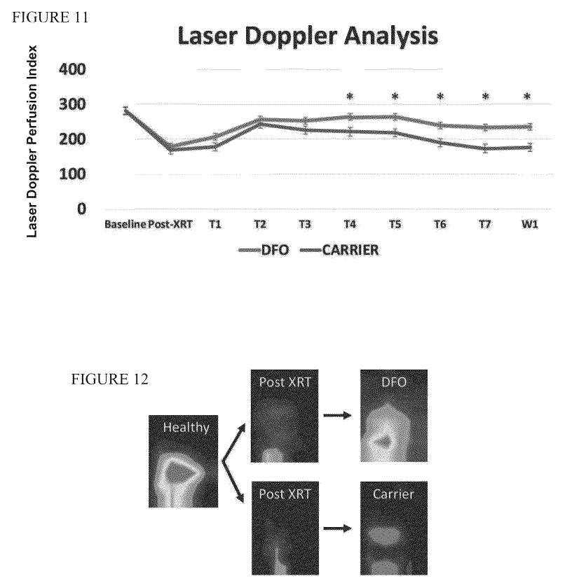

Laser Doppler Analysis (“LDA”) was performed prior to and after fat grafting to measure perfusion at the irradiated site, as described above in Example 1. FIGS. 11 and 12 show that mice with the DFO transdermal delivery patches (upper line in FIG. 11) showed significant improvements in blood flow (*p<0.05) compared to mice treated with transdermal delivery devices without DFO. FIG. 11 shows that quantification of laser Doppler perfusion index demonstrated scalp pretreated with a DFO transdermal delivery system (upper line) had significantly higher perfusion than scalp pretreated with a transdermal delivery system lacking DFO (lower line) one week after fat grafting (*p<0.05). FIG. 12 shows representative LDA images of mice scalp showing perfusion in mice prior to radiation therapy (leftmost image), in an experimental group pretreated with DFO delivered by a transdermal delivery device (upper two images), and in a control group pretreated with transdermal delivery devices without DFO (lower two images) immediately after cessation of radiation therapy and one week later. Darker areas represent lower perfusion and lighter areas represent higher perfusion.

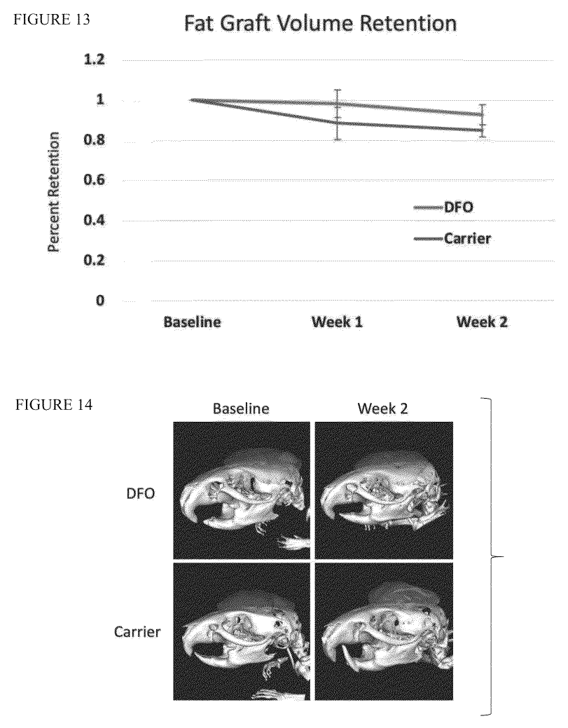

In vivo radiographic analysis of fat grafts showed DFO preconditioned irradiated mice retained more fat volume after two weeks compared to control mice (FIGS. 13-14). Fat graft volume retention was consistently greater among mice treated with transdermally administered DFO (upper line in FIG. 13) compared to control mice whose transdermal delivery devices lacked DFO (lower line in FIG. 13). FIG. 14 shows representative three-dimensional reconstructions of fat grafts after two weeks in either DFO (upper) or control (lower) preconditioned irradiated scalp.

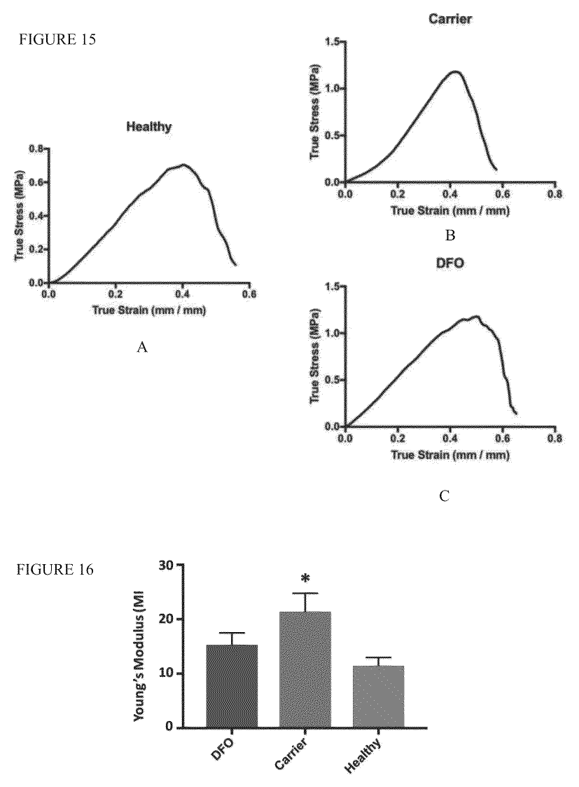

For skin analysis, scalp skin biopsy was harvested from both treatment groups following completion of radiation at the time of fat graft placement by trimming a piece of skin at the fat graft incision site. Scalp skin was also harvested from healthy mice that had not been irradiated. Skin stiffness was measured using a MTS Bionix 200 with an Interface SM-19 force transducer. Stress-strain curves were generated as shown in the figure and the Young's modulus (slope) was then calculated to figure out the stiffness. FIG. 15A shows the stress-strain curve for the healthy mice that had not been irradiated, FIG. 15B shows the stress-strain curve for the irradiated mice that had been treated with the transdermal delivery device without DFO, and FIG. 15C shows the stress-strain curve for the experimental group of irradiated mice treated with DFO via the transdermal delivery system. FIG. 16 summarizes the Young's Modulus data for the three groups. These data show that treatment of the skin with DFO after radiation therapy results in reduced skin stiffness.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of increasing retention of fat cells in a fat graft in irradiated tissue, the method comprising:
applying an effective amount of DFO to the irradiated tissue at a treatment site; and
increasing retention of fat cells in the fat graft.

2. The method of claim 1 wherein the applying step comprises injecting DFO under dermis at the treatment site.

3. The method of claim 1 wherein the applying step comprises applying DFO transdermally at the treatment site.

4. The method of claim 3 wherein the applying step comprises applying a transdermal delivery device to a tissue surface at the treatment site.

5. The method of claim 4 wherein the transdermal delivery system comprises DFO encapsulated in reverse micelles.

6. The method of claim 1 wherein the applying step comprises applying DFO to the irradiated tissue in multiple discrete doses.

7. The method of claim 1 further comprising grafting fat cells at the treatment site after the applying step.

8-19. (canceled)

20. A method of increasing collagen deposition in skin in irradiated tissue, the method comprising:
applying an effective amount of DFO to the irradiated tissue at a treatment site; and
increasing collagen deposition of the irradiated tissue at the treatment site.

21. The method of claim 20 wherein the applying step comprises injecting DFO under dermis at the treatment site.

22. The method of claim 20 wherein the applying step comprises applying DFO transdermally at the treatment site.

23. The method of claim 20 wherein the applying step comprises applying a transdermal delivery device to a tissue surface at the treatment site.

24. The method of claim 23 wherein the transdermal delivery system comprises DFO encapsulated in reverse micelles.

25. The method of claim 20 wherein the applying step comprises applying DFO to the irradiated tissue in multiple discrete doses.

26-31. (canceled)
